(19) United States
(12) Patent Application Publication
(10) Pub. No.: US 2023/0301566 A1
(43) Pub. Date: Sep. 28, 2023

(54) DEVICE, METHOD, AND SYSTEM FOR COLLECTION OF BLOOD

(71) Applicant: VITAL BIOSCIENCES, INC., Mississauga, Ontario (CA)

(72) Inventors: Paul Said Ehrlich Perez, Mississauga, Ontario (CA); Jackie GAO, Mississauga, Ontario (CA); Srinivas NADELLA, Mississauga, Ontario (CA); Famoud KAZEMZADEH, Mississauga, Ontario (CA)

(21) Appl. No.: 18/004,696

(22) PCT Filed: Jul. 13, 2021
(86) PCT No.: PCT/IB2021/056317

§ 371 Date: Jan. 9, 2023

Related U.S. Application Data
(60) Provisional application No. 63/051,562, filed on Jul. 14, 2020.

(51) Int. Cl. A61B 5/15

(52) U.S. Cl. CPC A61B 5/15003; A61B 5/150099; A61B 5/150412; A61B 5/150748

(57) ABSTRACT

A method and device for collecting a blood sample from a subject are provided. The handheld device comprises an actuator assembly and a body housing the actuator assembly and having a cavity configured to releasably receive a cartridge to couple to the actuator assembly. The cartridge is configured to capture the blood sample from the subject when used with the device. The blood sample is collected when the device determines that the cartridge is properly positioned over a vein or capillaries of the subject.

### CROSS REFERENCE TO RELATED APPLICATIONS

This application claims the benefit of U.S. Provisional Pat. Application No. 63/051,562 filed Jul. 14, 2020 entitled “Device, Method, and System for Collection of Blood”, which is incorporated by reference herein in its entirety.

### BACKGROUND

Blood sample and analysis are an indispensable part of patient’s diagnostics. Blood quality is the metric that is of utmost importance in clinical chemistry/pathology. Over 500 million blood specimens are collected annually in the US. Each blood draw is performed by a phlebotomist and the procedure, as well as follow-ups, are often inconvenient and time-consuming. For example, after the initial visit to the physician, the patients requiring a blood draw are often required to visit a secondary location for this service, adding time, inconvenience, and systemic costs.

Traditional methods of blood extraction are based on decades-old technologies such as the venipuncture (phlebotomy). But the phlebotomy process can be traumatic and inconvenient for some patients. In addition, the use of a hypodermic needle poses a risk of needle-stick injury, and may cause pain and anxiety in a patient. Some approaches, such as a finger prick (using a lancet), allow drawing blood without the need for phlebotomy. This method is the most common method for checking blood glucose levels. For neonates, a heal prick is used to extract small blood sample for a select few screening tests. The chief shortcoming of these methods is that the volume of blood extracted is limited by the amount of blood available in the capillary blood vessels that have been severed as a result of the lancing process, before the repair process is initiated by the body. Repeated squeezing (milking) can be used to slightly increase the volume of expelled blood but it is quite uncomfortable and laborious.

Some existing approaches to collecting capillary blood, as opposed to venous blood, allow collecting larger volumes of blood. Thus, some approaches allow creating several puncture wounds for collecting about 200 uL of blood from capillaries after several minutes of use. However, one of the concerns with testing capillary blood as opposed to venous blood is the fact that this method of extraction of blood has adverse effects on some blood parameters, which would then result in mis-diagnosis of a patient. The parameters that are most susceptible are white blood cells (WBC) count, red blood cells (RBC) count, platelet count, and potassium, more generally complete blood count (CBC) and electrolyte panels. The CBC and electrolyte panels are two of the most commonly requisitioned panels and these parameters are some of the most important parameters considered by physicians to determine the overall health of a patient. Thus, any deviation from the actual values can lead to misdiagnosis and therefore mistreatment of the patient.

Non-phlebotomy approaches to blood collection, as compared to phlebotomy-based approaches, are complicated due to the increase in WBC count (which can be caused by the body’s response to managing the wound as well as potential clumping of platelets that are mistakenly counted as WBCs), decrease in RBC count (the destruction of these fragile cells via the hemolysis process as a result of shear forces while the blood is being forced through the flesh wound), decrease in platelet count (these cells are responsible for blood coagulation and they clump and attempt to stop the bleeding when they come in contact with air and also as a result of shear forces as the blood is being forced through the flesh wound), and increase in potassium concentration (a side effect of hemolysis as red cells include a large amount of potassium inside which is not indicative of the true concentration of potassium).

In general, capillary blood collection methods have not been able to address the above issues and therefore have limited clinical utility as a general-purpose blood extraction method. In addition to the blood quality issues, lancing the finger may be an uncomfortable and painful process as there are many nerve endings at the tip of fingers. The amount of blood available for collection is also limited, which means that the finger will have to be “milked” in order to increase the sample volume, which reduces the quality of the extracted blood.

Accordingly, there is a need for improved devices and methods for collecting a blood sample from a patient.

### SUMMARY

In some aspects, the present disclosure provides a handheld device for collecting a blood sample from a subject. The handheld device comprises an actuator assembly and a body housing the actuator assembly and having a proximal end and a cavity configured to releasably receive a cartridge to couple to the actuator assembly, the cartridge being configured to capture the blood sample from the subject. The handheld device also comprises a handle coupled to the proximal end of the body, and a position detection system coupled to the body and having at least one sensor configured to determine a position of the cartridge relative to a target area of the subject when the cartridge is disposed within the cavity.

In some aspects, a cartridge for use with a handheld blood collection device is provided. In embodiments, the cartridge comprises a housing; at least one blood storage container contained within the housing; a distal body-contacting interface of the housing; a puncture assembly contained within the housing and comprising a puncture element moveable relative to the housing and contained within the housing in a first, retracted position and extending from the body-contacting interface in a second, extended position; and a blood drawing assembly coupled to the puncture element configured to collect a blood sample from a puncture created in a target area of a subject’s body by the puncture element in the extended position. The blood drawing assembly delivers the blood sample to the at least one blood storage container.

In some aspects, a method of collecting a blood sample from a subject is provided. The method comprises inserting a cartridge into a handheld device for collecting blood samples; positioning the handheld device with the cartridge against a forearm of a subject’s body; adjusting a position of the handheld device relative to the forearm until an indicator interface of the handheld device indicates that the cartridge is properly positioned over a vein in the forearm; pressing a body-contacting interface of the cartridge onto the forearm to stabilize the vein; actuating the handheld device thereby causing the cartridge to deploy a puncture element of a puncture assembly of the cartridge so that the puncture element moves from a first, retracted position to a second, extended position to create a puncture in the vein, wherein the actuating causes the cartridge to withdraw a blood sample through the puncture from the vein to at least one blood storage container included in the cartridge; when the blood sample is received in the at least one blood storage container, actuating the handheld device thereby causing the puncture element to move from the second, extended position to the first, retracted position; and separating the cartridge from the handheld device.

A puncture element of the puncture assembly can be a lancet or it can be a solid or hollow needle. In some embodiments, the needle can have perforations (e.g., through openings) forming various patterns on the needle’s wall. For example, the needle having a lumen can have one, two, or more perforations formed through its walls.

In some embodiments, a puncture element of the puncture assembly is in the form of an extendable needle that has a lumen extending therethrough. The needle is activated to move from the initial, retracted position to the second, extended position in which the needle creates a puncture in the subject’s body (e.g., in the vein). The needle then remains inserted into the puncture while the blood is being collected, such that the blood is delivered to the cartridge through the lumen of the needle.

In some embodiments, a puncture element of the puncture assembly is a lancet that is activated to move from the initial, retracted position to the second, extended position in which the lancet create a puncture in the subject’ vein and then returns to the initial position. Thus, the blood collection through the puncture is performed without any puncture element being inserted into the puncture.

### BRIEF DESCRIPTION OF THE DRAWINGS

Various features, objects, and advantages of the present invention will be described in connection with the accompanying drawings, which are incorporated in and constitute a part of this disclosure. The drawings illustrate exemplary embodiments of the invention and do not therefore limit its scope. In the drawings:

FIG. 1 is a perspective view of a handheld device in accordance with embodiments of the present disclosure, wherein the handheld device is shown with a removable cartridge inserted in the device.

FIG. 2 is a perspective view of the device of FIG. 1, wherein the cartridge is shown removed from the handheld device.

FIG. 3A is a perspective view of the cartridge of FIG. 1.

FIG. 3B is another perspective view of the cartridge of FIG. 1, showing a puncture element in an extended position.

FIG. 3C is a perspective view of a solid puncture element, showing the solid puncture element penetrating a skin surface.

FIG. 3D is a perspective view of a puncture element having a lumen extending therethrough, showing the solid puncture element penetrating a skin surface.

FIG. 4A is a side, perspective view of a cartridge in accordance with embodiments of the present disclosure.

FIG. 4B is a top, perspective view of the cartridge of FIG. 4A.

FIG. 4C is a side view of the cartridge of FIG. 4A.

FIG. 4D is a top view of the cartridge of FIG. 4A.

FIG. 4E is a bottom, perspective view of the cartridge of FIG. 4A.

FIG. 4F is a side, partially transparent view of a cartridge in accordance with embodiments of the present disclosure.

FIG. 4G is a side, partially transparent view of a cartridge in accordance with embodiments of the present disclosure illustrating a path that blood travels as it is collected using the cartridge.

FIG. 4H is another side, partially transparent view of a cartridge in accordance with embodiments of the present disclosure illustrating a path that blood travels as it is collected using the cartridge.

FIG. 4I is a side view of a blood storage container that is accessed from its top (proximal side) to obtain blood therefrom.

FIG. 4J is a side view of a blood storage container that is accessed from its bottom (distal end) to obtain blood therefrom.

FIG. 5A is a perspective view of a handheld device in accordance with embodiments of the present disclosure, wherein the handheld device is shown with a removable cartridge inserted in the device, and the device is inserted into a cradle.

FIG. 5B is a perspective view of the cradle of FIG. 5A.

FIG. 6A illustrates the handheld device of FIG. 1 in use, positioned over a forearm of a subject.

FIG. 6B is another view of the handheld device of FIG. 6A.

FIG. 7A is a perspective view of a tray with multiple compartments for storing cartridges.

FIG. 7B is a perspective view of the tray of FIG. 7A with the cartridges stored in the compartments, also showing the device of FIG. 1.

FIGS. 8A to 8F illustrate results of measurements of various blood parameters: WBC (while blood count) (FIG. 8A), Hematocrit (FIG. 8B), Platelets (FIG. 8C), Sodium (FIG. 8D), Chloride (FIG. 8E), and Potassium (FIG. 8F), using the present approach (“Rogue”) and the finger prick approach, as compared to standard phlebotomy.

### DETAILED DESCRIPTION

In some aspects, the present disclosure provides a handheld device for collecting a blood sample from a subject. The device is reusable, and it is used in conjunction with a removal disposable cartridge configured to create a puncture in a target area of a patient’s body (e.g., a vein in the forearm) and to withdraw the blood from the puncture. The cartridge has a puncture assembly and components of a blood drawing assembly for acquiring a blood sample and storing it in at least one blood storage container. A puncture element of the puncture assembly is disposed inside the cartridge and it is active only after the cartridge has been inserted into the handheld device and a trigger on the device is engaged.

The handheld device is easy to use and may be operated with no specialized training. The handheld device is able to detect a location of a vein on the patient’s forearm and allows precise targeting of a puncture assembly of the device. The handheld device has a position detection system that allows determining the position of the cartridge, or, more specifically, a puncture element, relative to the vein. In some embodiments, additionally or alternatively, the position detection system (comprising, e.g., one or more optical sensor) can be included in or associated with the cartridge. Also, the interface between the patient’s skin and the cartridge is designed to mechanically stabilize the vein in preparation for creating a puncture therein. This stabilization prevents the vein from moving away from the cartridge (more specifically, from the puncture element) and thus allows precise targeting of the vein. The vein may include any vein of a subject and/or capillary blood vessels of the subject.

The described device and cartridge allow for accurate, reliable, and minimally invasive blood withdrawal. The described approach is as efficient as traditional phlebotomy where the patient is only required to roll up their sleeve.

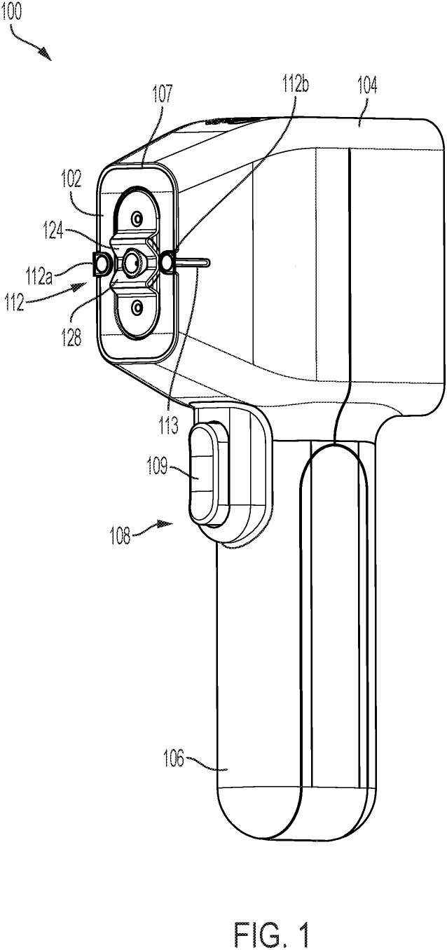

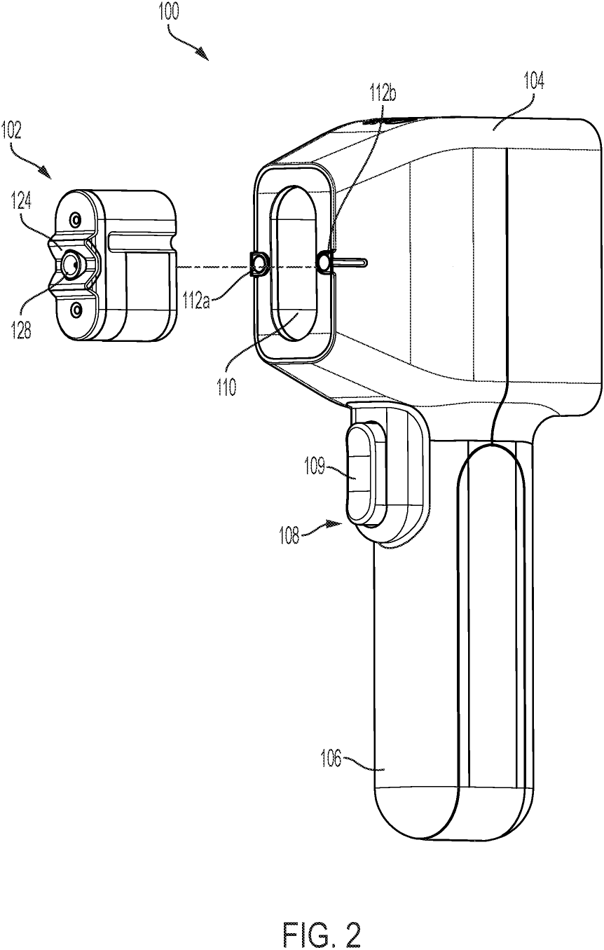

FIGS. 1 and 2 show an example of a handheld blood collection device 100 for drawing blood from a subject in accordance with embodiments of the present disclosure. The handheld device 100 can be a reusable device that is used in conjunction with a removable cartridge 102 that is configured to capture the blood sample from the subject. The cartridge 102 can be disposable. As discussed in more detail below, the cartridge 102 is used to puncture a subject’s body and to collect a blood sample from the subject’s body through the puncture, such that the collected blood is transferred into the cartridge. FIG. 1 illustrates the handheld device 100 with the cartridge 102 inserted therein, and FIG. 2 shows the handheld device 100 and the cartridge 102 separately.

As shown in FIGS. 1 and 2, the handheld device 100 comprises a body 104 having a proximal end, and a handle 106 coupled to the proximal end of the body. The handle 106 can have a grip portion and other features that allow it to be conventicle held by a user. The body 104 of the handheld device 100 houses an actuator assembly 108 and has a cavity 110 (visible in FIG. 2) configured to releasably receive the cartridge 102 to couple to the actuator assembly 108. In the illustrated embodiment, the cavity 110 is formed in a distal portion of the body 104 that faces a subject’s body in use. It should be appreciated however that the cavity 110 can be formed in other ways in the body 104 of the device 100. Regardless of the specific position and configuration of the cavity, it is formed such that it releasably fits the cartridge 102.

The actuator assembly 108 comprises an actuator 109 (e.g., a button, switch, or pad) disposed on the handle 106 and configured to be activated by a user action, e.g., by pressure applied thereto, as discussed in more detail below. It should be appreciated that the actuator can be in any other form. It can be activated by the user pressing it or, depending on its configuration, in other ways.

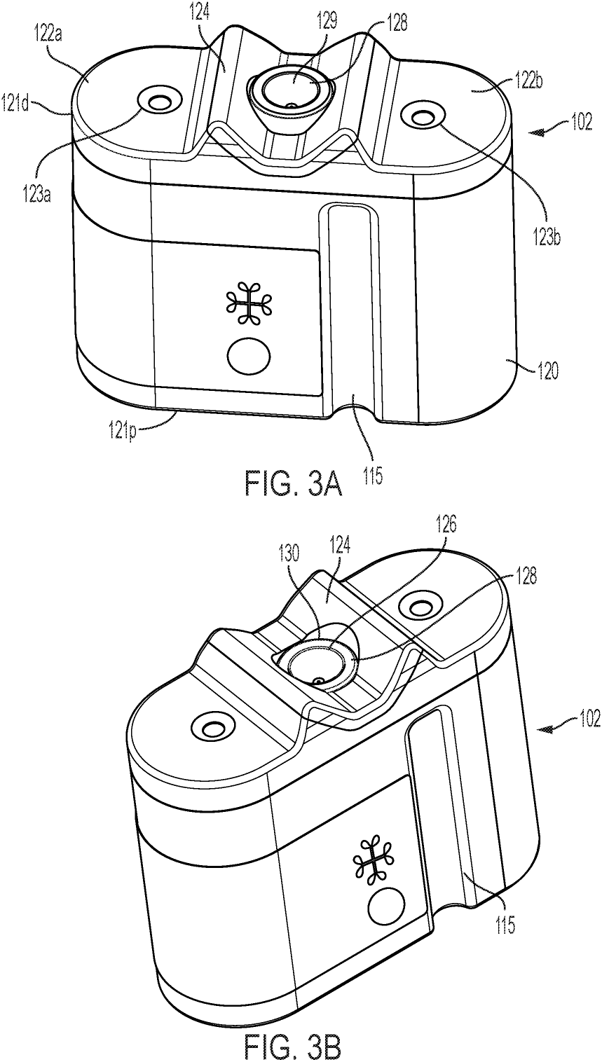

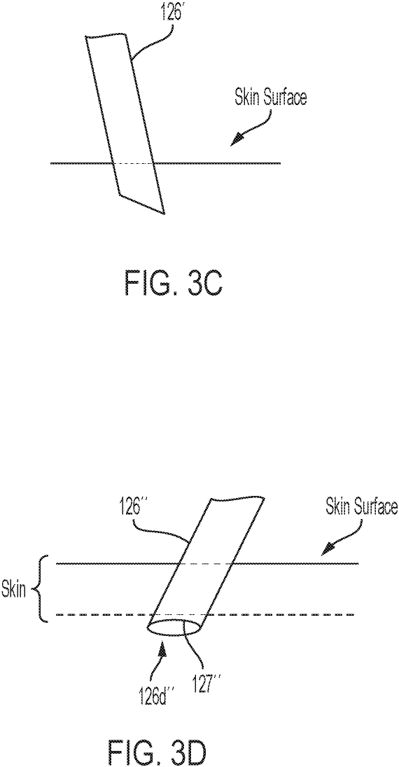

In embodiments of the present disclosure, in use, the cartridge 102 is inserted into the cavity 110 of the device’s body 104 such that the cartridge 102 is partially exposed and operably couples with the device 100 to position the puncture element 126 (see FIG. 3B) relative to the patient and control actuation of the needle and blood collection. The process of blood sample collection using the cartridge 102 is thus controlled via the device.

In order to more accurately and efficiently position the puncture element 126 relative to the patient, the handheld device 100 comprises a position detection system 112 coupled to the body 104 of the device 100 and has at least one sensor configured to determine a position of the cartridge 102 relative to a target area of the subject when the cartridge 102 is disposed within the cavity 110. In the illustrated implementation, the position detection system 112 includes first and second optical sensors 112a, 112b positioned on a distal side of the body, proximate to opposite sides of the cavity. The handheld device 100 can have other components and features that assist in proper positioning of the puncture element for blood withdrawal. Thus, as shown in FIGS. 1 and 2, the body 104 of device 100 may include indicia such as grooves or lines extending substantially perpendicular to a longitudinal axis of the handheld device 100 that denote the location of the first and second optical sensors 112a, 112b. One of such lines, line 113, demarking the location of the sensor 112b is shown in FIGS. 1 and 2. The identical line, demarking the location of the sensor 112a, is obscured in FIGS. 1 and 2.

In some embodiments, the position detection system can include components that assist the user operating the device 100 in determining whether the cartridge 102, inserted in the device 100, is properly positioned over the target area of the subject’s body, e.g., over a vein in the subject’s forearm. For example, in some embodiments, the handheld device includes an indicator interface that provides a visual indication of the position of the cartridge 102 relative to the target area, which includes the indication of the proper, ready-to-deploy, position of the cartridge 102.

The position detection system can be activated in various ways. In some embodiments, it is activated automatically when the handheld device 100 detects that the cartridge 102 is received in the cavity 110 and coupled to the actuator assembly.

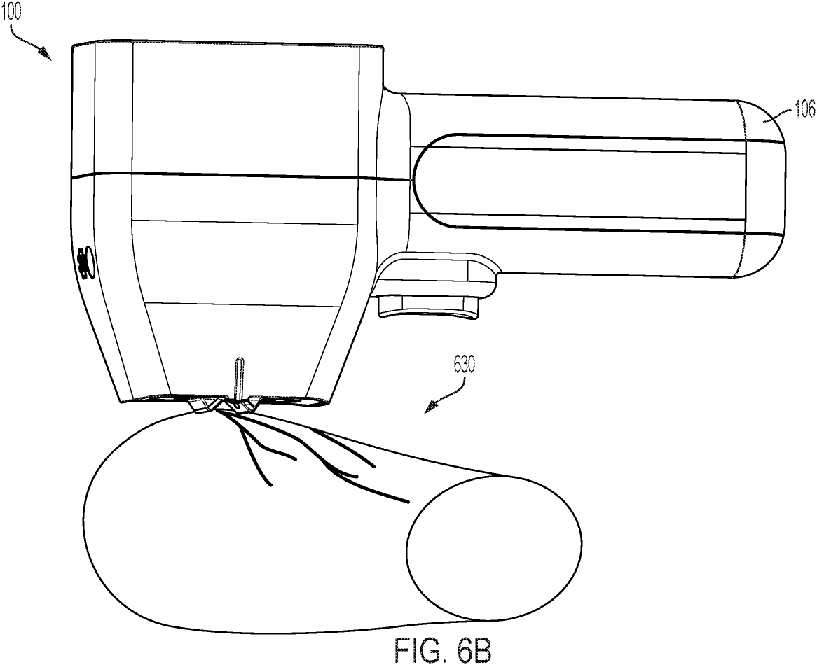

Thus, FIG. 6A shows the indicator interface of the position detection system such as a display or graphical user interface 300 configured to present a graphical indication of the position of the cartridge 102 relative to the target area. The graphical user interface 300 can be disposed on the proximal side 105p of the body 104 of the device 100 having proximal and distal sides 105p, 105d, as shown in FIG. 6A. As also shown in FIG. 6A, when the cartridge 102 is positioned over the target area, such as the vein, the graphical user interface 300 displays a first line corresponding to a position of a first portion of the vein and a second line corresponding to a position of a second portion of the vein. In use, a position of the handheld device relative to the forearm is adjusted until the first and second lines are aligned (e.g., are on the same axis), which indicates that the cartridge is properly positioned over the vein. In this way, the graphical user interface 300 allows the user to determine whether or not the cartridge 102 is properly positioned for deployment.

The graphical user interface 300 can have any other visual indicators that facilitate the use of the device 100. For example, as shown in FIG. 6A, the graphical user interface 300 includes the “Ready” visual indicator that additionally indicates to the user that the cartridge 102 is in the proper position for firing the puncturing element into the vein. It should be appreciated that such indicator is shown by way of example only, as any suitable words, phrases, signs, and/or symbols can be displayed (and they can be in any language). Furthermore, it should be appreciated that the position detection system can have any other component(s) that facilitate determining or determine the position of the cartridge relative to the target area, such as the vein in the subject’s forearm. Also, the components of the position detection system described herein can be implemented in various ways. For example, in some implementations, in addition or alternatively to the visual indicators of the cartridge’s position, of the position detection system can generate an audio signal indicating the proper position of the cartridge.

As shown in FIGS. 1 and 2, and also in FIGS. 3A and 3B that illustrate the cartridge 102, the cartridge 102 comprises a housing 120 that includes various components of the cartridge 102. Thus, the housing 120 includes at least one blood storage container (such as blood storage containers 122a, 122b), a distal body-contacting interface 124 of the housing 120, and an isolation chamber 128 coupled to the housing 120.

The housing 120 can also include components for removable attachment of the cartridge 102 to the device 100. Thus, as shown in FIGS. 3A and 3B, in this example, an outer surface of the housing 120 has at least one longitudinal groove 115 extending from a proximal end 121p of the housing 120 towards a distal end 121d of the housing 120 and configured to mate with a corresponding protrusion formed on an inner surface of the cavity 110 (obscured). The housing 120 can have a groove, such as the longitudinal groove 115, formed on each of its sides. The grooves formed in the housing 120 thus mate with corresponding protrusions when the cartridge 102 is inserted into the device 100. It should be appreciated however that the device 100 and cartridge 102 can have any other features that allow for reversible coupling between the cartridge 102 and the device 100, as the groove 115 is shown as an example only. In some embodiments, one or more magnets can be used to hold the cartridge at least partially inside the device. Any other external features (e.g., formed on the device’s housing and/or on the cartridge’s housing) or internal features can be used to reversibly mate the cartridge to the device. The features can include release feature(s) configured to be activated to disengage the cartridge from the device after use.

In the embodiment illustrated in FIGS. 3A and 3B, the cartridge 102 can be inserted into the cavity 110 and removed therefrom, as it slides along the protrusions on the inner surface of the cavity 110. The longitudinal groove 115 is shown by way of example, as one or more other attachment elements, of any suitable size and configuration, can be formed in and/or on the housing 120. The attachment elements can be formed in the surface of the housing or they can be coupled to the housing 120. The longitudinal groove(s) 115 and/or other attachment elements of the cartridge 102 can be releasably mated with corresponding features within the cavity 110 such that the cartridge 102 securely remains in place while in use, until the blood sample is collected and the cartridge 102 is separated from the device 100.

Also, the cartridge 102, for example, the proximal end 121p of the housing 120, has contact elements that mate with the interior of the cavity 110 of the device 100. Thus, the cartridge 102 operably couples to the actuator assembly 108 of the device 100 (e.g., to its circuity) such that operation of the cartridge 102 is controlled via the actuator assembly 108.

In the illustrated embodiments, the body-contacting interface 124 is a grooved channel having a shape that corresponds to a shape of the target area of the subject. In the example illustrated, the grooved channel is generally V-shaped, though it should be appreciated that the channel can have any other concave shape such as, e.g., C or U shaped. In use, prior to activating the handheld device 100 to cause the cartridge 102 to create a puncture in the target area of the subject’s body, the body-contacting interface 124 of the cartridge 102 is pressed onto the target area to stabilize that area. In embodiments, the target area is the subject’s forearm and the body-contacting interface 124 is pressed onto the body-contacting interface 124 to stabilize a vein in the forearm before the vein is punctured for blood withdrawal. In some embodiments, the body-contacting interface can be formed of at least partially compressible material, which facilitates the stabilization of the target area when the cartridge 102 inserted into the device 100 is positioned over and pressed onto the target area.

The handheld device 100 can also have features that facilitate the contact of the cartridge 102 inserted into the device 100 with the surface of the target area of the subject’s body, such as the forearm. Thus, as shown in FIGS. 1 and 2, a portion 107 of the body 104 of the device 100 proximate to the cavity 110 has curved skin contacting edges. Thus, the distal portion of the body 104 is shaped so as to conveniently facilitate firm contact between the cartridge 102 and the target area, while also ensuring the subject’s comfort during the blood withdrawal procedure.

The cartridge 102 also comprises a puncture assembly contained within the housing 120 and comprising a puncture element 126 moveable relative to the housing 120. The puncture assembly, such as, e.g., a lancet assembly, allows puncturing a target area of a subject’s body (e.g., a vein in the forearm) to create a puncture in the target area. In some embodiments, the cartridge 102 includes a plurality of puncture elements 126 to allow the puncture assembly to create a plurality of punctures at a target area of the subject’s body. As shown in FIGS. 3A and 3B, the puncture assembly comprises a puncture element 126, such as a lancet or needle (which can be solid or hollow), that is configured to move from an initial (also referred to herein as “first”) retracted position to an extended (“second”) position. Thus, in FIG. 3A, the puncture element 126 is positioned within the housing 120 of the cartridge 102, such that the puncture element 126 is in the first, retracted position and therefore not visible. FIG. 3B illustrates the puncture element 126 in the second, extended position, in which the puncture element 126 extends distally from the surface of the cartridge’s housing 120. In the extended position, the puncture element 126 extends from the body-contacting interface 124 of the housing 120. The handheld device 100 may be configured to control the amount that the needle that extends distally from the surface of the cartridge’s housing 120, when the needle is in the extended position. Thus, the depth of the needle in a subject, when the needle is in the extended position, may be controlled by the handheld device 100.

In some embodiments, a puncture element can have various forms. For example, it can be a hollow or solid needle, or a lancet. In some embodiments, the needle can have perforations (e.g., through openings) forming various patterns on the needle’s wall. The perforations can facilitate withdrawal of blood from the vein. In some embodiments, the needle (with perforations or without) is associated with features that allow detection that the needle has entered the vein and detection that the needle has encountered liquid. The needle can thus be able to detect contact with liquid using one or more of electrical impedance, capacitance, or conductance. The cutting/leading edge at a distal end of the puncture element can have any suitable shape, including an angled shape (e.g., V-shape) and/or a guillotine-type shape, a star shape, or a cross shape. In some embodiments, the needle is comprised of a plastic material. In one embodiment, the needle is comprised of a translucent material to allow for an optical wave guide to extend through the needle such that a user may view the wave guide to assist with locating a subject’s vein.

FIG. 3C shows an example of a puncture element in the form of a solid needle 126′ that is shown penetrating, with its distal end, a subject’s skin surface to access blood inside a vein. The needle 126′ can be extended to create a puncture in the skin in an extended configuration and then retracted once the puncture is created. A puncture element in the form of a lancet can similarly be used to create a puncture in an extended configuration and then retracted into the initial configuration once the puncture is created.

In some embodiments, a puncture element of the puncture assembly is in the form of an extendable needle that has a lumen extending therethrough. FIG. 3D shows an example of a puncture element in the form of a needle 126″ having a lumen 127″ extending therethrough that can receive blood during blood collection. In FIG. 3D, the needle 126″ is shown inserted through the skin surface such that its distal end 126d″ extends partially or entirely through the skin layer, to access the vein (not shown) underneath the skin layer. The needle 126″ is activated to move from the first, retracted configuration to the second, extended configuration in which it punctures the skin and penetrates the vein to access blood. The needle 126″ remains in the extended configuration while the blood is collected and the blood is provided to the cartridge through the lumen 127″ of the needle 126″.

In embodiments, a gauge of a needle or another puncture element can be in the range of from 17 mm to 22 mm. In some embodiments, the device 100 and/or the cartridge 102 includes a mechanism to control a penetration depth of the needle. The penetration depth of the needle or lancet can be controlled to be from about 2 mm to about 10 mm. In some embodiments, the penetration depth can be about 7 mm. The penetration depth can be measured using a position detection system or another system employing one or more optical sensors.

In some embodiments, a puncture element of the puncture assembly can include or can be associated with liquid detection sensor(s) that allow detecting when the puncture element reaches blood as it is moved through the skin. In such embodiments, for example, with reference to FIG. 3D, it can be detected that the puncture element 126″ has passed through the skin and its distal end 126d″ has reached the blood. The sensor can be a capacitance sensor, an impedance sensor, or a conductance sensor. It should be appreciated that embodiments of the present disclosure are not limited to any specific type of sensor configured to sense blood or another liquid.

In addition, in some embodiments, a puncture assembly of a cartridge can include more than one puncturing element 126 (e.g., more than one needle).

Referring back to FIGS. 3A and 3B, the cartridge’s housing 120 includes a blood drawing assembly configured to collect the blood sample from a puncture created in the target area by the puncture element 126 in the extended position. The blood drawing assembly delivers the collected blood sample to the blood storage containers 122a, 122b. In some embodiments, the blood drawing assembly can be coupled to the puncture assembly.

In embodiments of the present disclosure, the cartridge 102 includes components that facilitate blood withdrawal from the vein. Thus, as mentioned above, and as shown in FIGS. 1, 2, 3A, and 3B, the cartridge 102 comprises an isolation chamber 128 coupled to the housing 120. The body-contacting interface 124 comprises an opening 130 (see FIG. 3B) that receives therethrough the isolation chamber 128. The isolation chamber 128 can be generally conical such that its diameter increases in a distal direction. The isolation chamber 128 has a central opening or passage that receives therethrough the puncture element 126 in the extended position, as shown in FIG. 3B. In embodiments, the isolation chamber 128 can be retractable such that it is retracted proximally towards the housing 120 when the puncture element 126 moves from the initial position to the extended position. Thus, in FIG. 3A, the isolation chamber 128 is shown in the original position in which a distal rim 129 of the isolation chamber 128 extends at a distance from the opening in the body-contacting interface 124. In this configuration, as mentioned above, the puncture element 126 is in the initial position and it is disposed within the housing 120. As the puncture element 126 moves from the initial position to the extended position, the isolation chamber 128 is retracted proximally towards the housing 120, as shown in FIG. 3B. In such configuration, the isolation chamber 128 sits deeper within the opening 130 in the body-contacting interface 124. In use, the isolation chamber 128 is positioned around the puncture created by the puncture element 126 in the extended position and creates a seal around the puncture to assist in blood withdrawal through the puncture while preventing the puncture from prematurely closing, as discussed in more detail below. In some embodiments, after blood is collected and the cartridge is withdrawn away from the puncture in the subject’s skin, the isolation chamber 128 is positioned so that it protects the puncture element. Such configuration allows keeping the puncture element (e.g., without limitation, a needle) in a safe position in which the isolation chamber 128 at least partially “hides” the puncture element, particularly its sharp end.

It should be appreciated that the isolation chamber 128 is shown in FIGS. 3A and 3B by way of example, and that the isolation chamber can have other configurations and sizes, and it can be positioned in a suitable way with respect to the puncture element.

The puncture element 126 of the puncture assembly is activated using the actuator assembly of the handheld device. Referring back to FIGS. 1 and 2, the actuator assembly 108 is configured to be activated to cause the puncture element 126 to puncture a skin surface of the subject at the target area and to cause the blood withdrawal assembly to collect the blood sample from the target area through the puncture. For example, the actuator 109 (e.g., a button) disposed on the handle of the handheld device can be pressed to activate the puncture element 126. The activation can occur after it has been determined e.g., by the position detection system, that the device (the cartridge inserted in the device, to be exact) is properly positioned with respect to the target area, e.g., the vein, and the vein is stabilized.

In embodiments, the actuator assembly 108 configured to apply negative pressure to the target area while the blood withdrawal assembly draws the blood sample through the puncture in the target area. The negative pressure is applied to the puncture wound having the isolation chamber 128 disposed therein such that, as the blood is sucked into the cartridge, the skin at the target area adopts a dome-like shape with the puncture wound (which is stretched open) at the apex. The application of the negative pressure to the puncture thus assists in maintaining the puncture open during the blood withdrawal process. This also affects positively the quality of the withdrawn blood.

Referring back to FIGS. 3A and 3B, the blood drawing assembly delivers the blood sample, as the blood is being collected, to the blood storage containers 122a, 122b. The blood drawing assembly can comprise at least one fluid conduit (e.g., one or more tubes, not shown) configured to deliver the blood sample acquired through the puncture to the blood storage containers 122a, 122b. The blood drawing assembly can include a pump (positioned in the handheld device) and any other suitable component(s). Each of the blood storage containers includes an access port for removing the blood sample from that blood storage container. Thus, as shown in FIGS. 3A and 3B, the blood storage containers 122a, 122b have access ports 123a, 123b, respectively. In use, after the blood sample is transferred into the blood storage containers and the cartridge 102 is removed from the device 100, the blood storage containers 122a, 122b can be accessed via the ports 123a, 123b to remove the blood therefrom, for analysis. In some embodiments, one or more of the blood storage containers 122a, 122b are removable from the cartridge housing 120.

It should be appreciated that two blood storage containers 122a, 122b are shown by way of example only, as the cartridge can have a single blood storage container or more than two blood storage containers (e.g., three, four, or more than four blood storage containers). Also, the one or more blood storage containers can be disposed in various ways within the cartridge, and the containers can be accessed in various ways as the present disclosure is not limited in this respect. The blood storage containers of the cartridge can have the same or different configuration.

In some embodiments, the blood storage container(s) (or vials) can be plastic vessels which are coated with blood anticoagulants and preservatives, e.g., k2EDTA and Lithium Heparin. The coating can be spray coating or any other technique. In some embodiments, each blood storage container can carry a maximum volume of about 600 uL of blood. Blood storage containers configured to hold a suitable volume of blood, including other than 600 uL, can be used as well.

The handheld device 100 can have other components and features that assist in skin puncture and blood withdrawal. For example, in some embodiments, the device 100 has a depth control actuator coupled to the body 104 and configured to be actuated to control a depth of the puncture to be created by the puncture element 126 in the extended position. The depth control actuator can be in the form of a dial, button, switch, or another device configured to be manipulated to adjust the desired depth of the penetration. The depth control actuator can be disposed on the handle 106 of the device in some implementations. Also, in some embodiments, instead of using a depth control actuator configured to receive used input, the depth of the puncture can be controlled automatically, e.g., via a computing device that controls operation of the device 100.

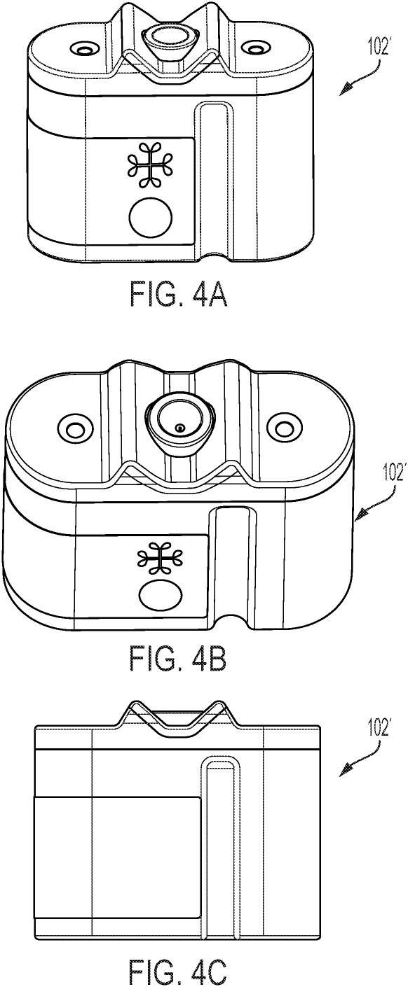

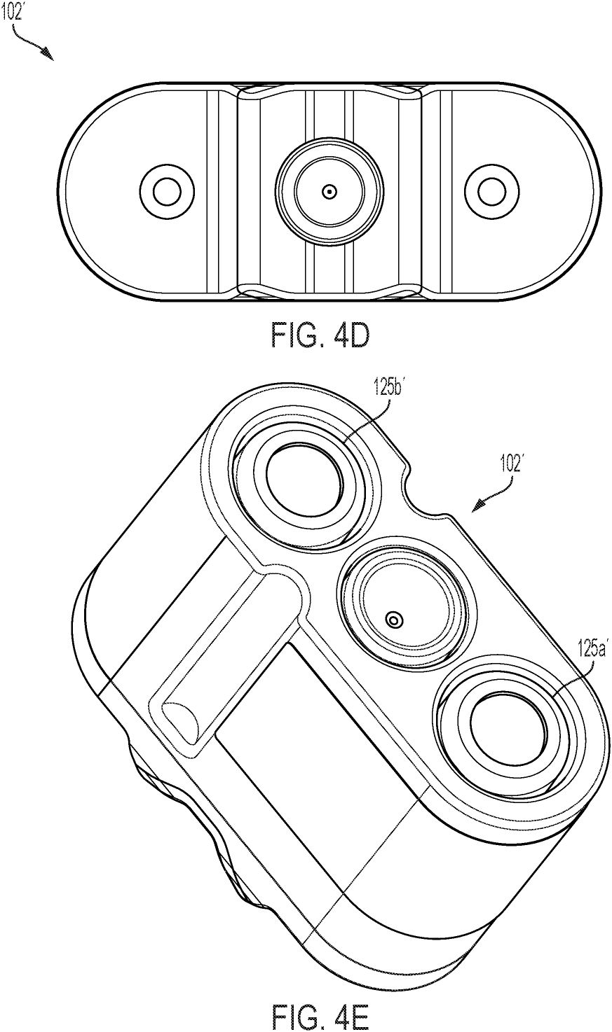

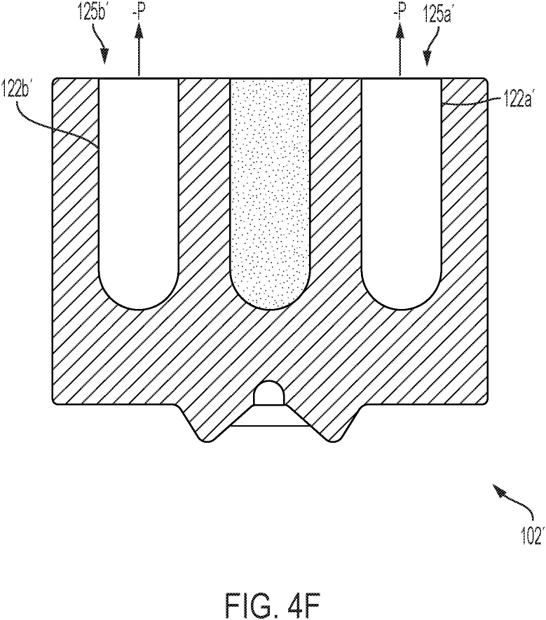

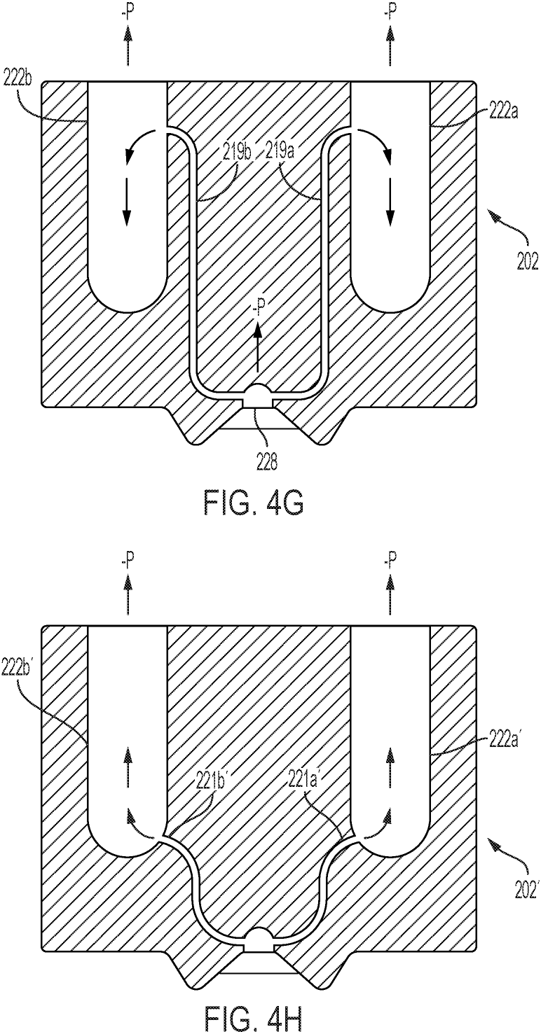

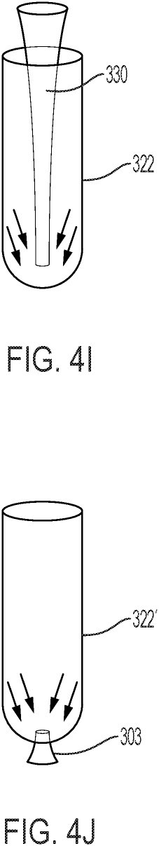

FIGS. 4A, 4B, 4C, 4D, and 4E illustrate several views of a cartridge 102′ which is similar to the cartridge 102 shown in FIGS. 1, 2, 3A, and 3B.

In embodiments, blood storage containers can have various configurations. In some embodiments, for example, they can be configured as tubes which can have various shapes. For example, the tubes can have conical or rounded tips, or tips having other shapes. Other configurations can be used additionally or alternatively.

Blood can be transmitted to the interior of the blood storage containers in various ways. In some embodiments, for example, blood storage containers can have one or more openings in their walls which can be in fluid communication with fluid conduits (e.g., tubes) that deliver blood from a puncture to the blood storage containers. Each of the blood storage containers can have one or more openings in fluid communication with fluid conduits, and the opening(s) can be disposed in various locations on the blood storage containers, at various distances from a distal end of the cartridge.

FIG. 4G illustrates an example of a cartridge 202 (which can be similar to cartridge 102 in FIGS. 3A and 3B) that is operably coupled to a handheld device (not shown) in accordance with embodiments of the present disclosure. As shown in FIG. 4G, the cartridge 202 has blood storage containers 222a, 222b, each of which has an opening that communicates with a respective tube or fluid conduit through which the blood is delivered from the puncture to the blood storage containers 222a, 222b. After a puncture element (not shown), having an isolation chamber 228 disposed therearound, creates the puncture in the vein, constant negative pressure (shown as “-P” in FIG. 4G) is applied by the handheld device which causes the blood to be delivered from the puncture via the fluid conduits 219a, 219b to the blood storage containers 222a, 222b, respectively (as also shown by arrows drawn within the blood storage containers). In the example of FIG. 4G, the constant negative pressure is applied at the blood storage containers 222a, 222b, as well as at the isolation chamber 228. The constant negative pressure is applied by a blood drawing assembly, which can have parts (e.g., fluid conduits) disposed in the cartridge and some parts (e.g., a pump) positioned in the handheld device.

FIG. 4H shows another example of a cartridge 202′ that can be similar to cartridge 202 of FIG. 4G. In FIG. 4H, respective openings 221a′, 221b′ in blood storage containers 222a′, 222b′ of the cartridge 202′, in fluid communication with respective fluid conduits, are positioned more distally along the length of the blood storage containers than the openings in the blood storage containers of FIG. 4G. In this way, depending on the position of the openings, the blood storage containers are filled from their bottoms (as shown in FIG. 4H) or closer to the tops (FIG. 4G). In the example of FIG. 4H, the constant negative pressure is applied to the blood storage containers 222a′, 222b′.

Furthermore, in some embodiments, blood storage containers of a cartridge in accordance with embodiments of the present disclosure can have more than one fluid conduit connected thereto, and the fluid conduits can be disposed at different positions along a length of the blood storage containers. Furthermore, in embodiments in which the cartridge has two or more blood storage containers, the blood storage containers can have different number of fluid conduits (tubes) coupled thereto, and the fluid conduits can be coupled to the blood storage containers at different positions along the length and/or other dimensions of the blood storage containers.

In embodiments, proximal ends of the blood storage containers can have membranes, e.g, hydrophobic membranes. FIG. 4E shows an example of an implementation of a bottom, or proximal side, of the cartridge 102′. In FIG. 4E, proximal sides 125a′, 125b′ of blood storage containers 122a′, 122b′ (which are similar to blood storage containers 122a, 122b) are shown, respectively. The proximal sides 125a′, 125b′ can each include a hydrophobic membrane, as shown schematically in FIG. 4F illustrating blood storage containers 122a′, 122b′. The blood storage containers can have their proximal sides formed from hydrophobic membranes made of suitable materials. In this way, the vacuum can be created through the back of the blood storage container(s), but no blood would seep through the membranes as the blood is being withdrawn.

The handheld device 100 in accordance with the present disclosure can be configured such that a constant negative pressure (shown as -P in FIG. 4F) is created and, as air is pulled proximally through the blood storage containers, the blood is acquired from a subject’s vein and is delivered to the blood storage containers. The actuator assembly 108 of the handheld device 100 may include a pump (e.g., a vacuum pump) that operates to create the constant negative pressure that allows the blood to be withdrawn from the puncture in the subject’s vein. In some embodiments, the vacuum created in the blood storage container(s) has a corresponding vacuum pressure and the actuator assembly 108 of the handheld device 100 is configured to modulate (e.g., increase or decrease) the vacuum pressure. In some embodiments, the vacuum pump is configured to modulate the vacuum pressure. By modulating the vacuum pressure, a user may experience a rubbing or kneading sensation (e.g., a massaging sensation) on the area of the subject’s body proximate the puncture in the subject’s vein, which may provide additional comfort to the user and improve the withdrawal of blood from the puncture in the subject’s vein. For example, the actuator assembly 108 may increase the vacuum pressure to a first predetermined pressure and then decrease the vacuum pressure to a second predetermined pressure, or vice versa, one or more times to cause the user to experience a kneading sensation on the area of the subject’s body proximate the puncture in the subject’s vein. In some embodiments, the first predetermined pressure is about -7 psi and the second predetermined pressure is about -2 psi. In some embodiments, the increasing and decreasing of the vacuum pressure from the first predetermined pressure to the second predetermined pressure may be done gradually over a period of time (e.g., one second, two seconds, three seconds, four seconds, five seconds, or more than five seconds).

FIG. 4F also illustrates that a distal end of a blood storage container can be generally conical. Such shape allows minimizing “dead” volume of blood (blood pooling) that may otherwise accumulate in the storage container.

Blood storage containers have features that allow accessing blood therefrom. For example, as discussed above and shown in FIGS. 3A and 3B, blood storage containers have access ports, such as access ports 123a, 123b, respectively. Blood in the blood storage containers can be accessed for analysis in various ways. For example, as shown schematically in FIG. 4I, a blood storage container 322 can be accessed from the top (its proximal end) using a suitable element, such as, e.g., without limitation a pipette 330 shown in FIG. 4I.

In some embodiments, after the blood is collected and stored in the blood storage containers, as the cartridge with the blood storage containers is transferred from the handheld device to an analyzer (e.g., to a tray or receptacle of the analyzer), the access ports can be pierced through. In such embodiments, the receptacle, configured to receive the cartridge with the collected blood therein, can have piercing features with a sharp end that are disposed on the receptacle such that, as the cartridge is placed onto the receptacle, the piercing features are aligned with the blood storage containers’ access ports and the access ports are pierced to access the blood stored therein. The blood can thus be transferred to a receptacle. FIG. 4J shows schematically a blood storage container 322′ that can have its distal end pierced by a piercing element 303 (e.g., a needle) positioned in a tray or receptacle of an analyzer.

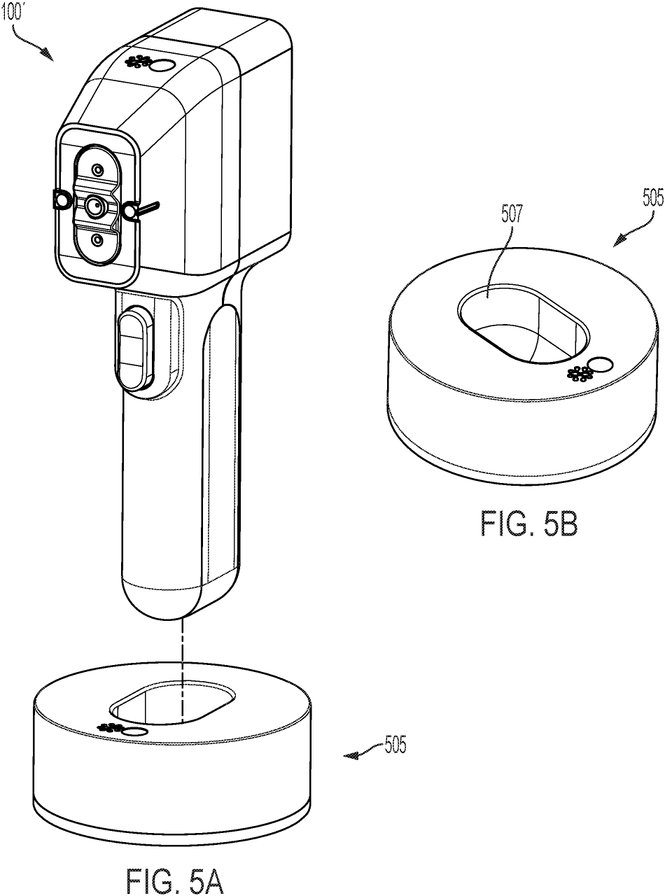

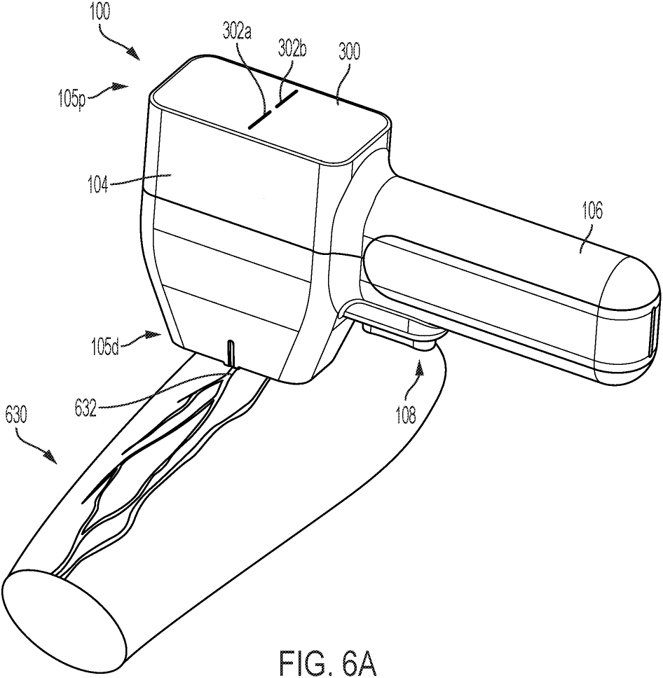

FIGS. 5A and 5B illustrate a handheld device 100′ having the cartridge 102′ inserted therein. As shown in FIG. 5A, a distal end of a handle 106′ of the device 100′ can couple with a charging cradle 505 that charges the device 100′. The cradle 205 can have an opening 507 (FIG. 5B) configured to removably seat therein the distal end of the handle 106′. It should be appreciated that the cradle 505 is shown as generally circular by way of example only, as it can have any other shape and configuration.

Regardless of its specification configuration, a handheld device, configured to receive a removable cartridge for use with the device in blood withdrawal from subjects, is used for collecting a blood sample from a subject in an improved manner. In particular, as discussed above, the present approach is as convenient as phlebotomy such that the patient is only required to expose his/her forearm. Moreover, the present device allows to physically trap the vein (which can naturally “move around”) to thereby limit its range of motion. By thus trapping the vein and stabilizing it in place, the likelihood of accessing the vein from a single attempt for withdrawal of the desired volume of blood is increased. The present approach is thus more reliable and more convenient to a patient and to a user (medical personnel) as compared to the standard phlebotomy. Also, as discussed above, the application of the negative pressure to the puncture wound as blood is being withdrawn therefrom allows keeping the puncture open during the blood withdrawal, which in turn allows reducing the amount of sheer stress imposed on the blood during its withdrawal.

Furthermore, because of the ease of the use of the device in accordance with embodiments of the present disclosure, it can be used by a healthcare professional (e.g., nurse, medical assistant, technician, etc.) with minimal training related to the device operation. In this way, the device can be used in any setting, including settings where a highly skilled trained professional (phlebotomist) is not present.

As discussed above, the handheld device is reusable, whereas the cartridge is disposable. The cartridge can be picked up with the device (i.e. directly from a manufacturing package), as discussed in more detail below, and used for blood extraction from a patient. After the blood sample is acquired, the cartridge is removed from the device and is transferred to an analyzer or stored or shipped to another location. The sample collection device can comprise a plurality of compartments or receptacles each configured to fit a cartridge. In some embodiments, the handheld device is configured to be attached or otherwise coupled to a subject (e.g., via one or more straps) rather than be held by a user when in use.

It should be appreciated however that, in some embodiments, the cartridge can be reusable. The cartridge may be configured to be recycled/refurbished. For example, the cartridge may have removable reservoirs (blood storage containers) and, after cleaning and sterilizing, and installing new reservoirs, the cartridge may be reused.

In some aspects, a method of collecting a blood sample from a subject is provided. The method comprises inserting a cartridge into a handheld device for collecting blood sample. For example, device 100 (FIGS. 1 and 2) can be used, which receives therein cartridge 102 (FIGS. 1, 2, 3A, and 3B). As shown in FIGS. 6A and 6B (illustrating device 100 and cartridge 102), the method further includes positioning the handheld device with the cartridge against a forearm 630 (shown very schematically) of a subject’s body and adjusting a position of the device relative to the forearm until an indicator interface of the handheld device indicates that the cartridge is properly positioned over a vein 632 in the forearm 630. As discussed above, the indicator interface can be, e.g., the graphical user interface 300 of the position detection system of the device 100 illustrated in FIG. 6A. The indicator interface is coupled to at least one optical sensor of the handheld device that determines a location of the vein. The position detection system, such as, in some implementations, at least two optical sensors, allows detecting the location of a superficial vein on the patient’s forearm and allows for precise targeting of the puncture assembly. The optical sensors sense absorption due to hemoglobin in the blood. In some implementations, the device 100 can perform stereo-like measurement to measure the approximate depth of the vein under the skin surface.

In the illustrated example, with reference to FIG. 6A, the graphical user interface 300 displays a first line 302a corresponding to a position of a first portion of the vein 632 and a second line 302b corresponding to a position of a second portion of the vein 632. The position of the handheld device 100 relative to the forearm 630 is adjusted until the first and second lines 302a, 302b are aligned (e.g., are on the same axis), which indicates that the cartridge 102 is properly positioned over the vein 632. It should be appreciated that the graphical user interface 300 in FIG. 6A is shown by way of example only. Accordingly, the first and second lines 302a, 302b are only an example of an implementation of indicator features that are indicative of the proper position of the device for puncturing the vein. For example, indicator feature(s) can include lights of different colors, e.g., red, yellow/orange, and green, with red indicating that the device is not positioned properly, and green indicating a proper position. As another example, the indicator features can include a blinking light indicator (e.g., the indicator can blink until a proper device position is detected). The device can be implemented to generate any suitable visual and/or audio signals as indicator(s) of a current position of the device and indicator(s) of a device’s position with respect to the subject’s body in which the device can be operated to puncture the vein to acquire blood therefrom.

In some embodiments, the position detection system can be activated automatically, once the cartridge is inserted into the device. The device can sense that the cartridge is connected thereto, and the position detection system can begin scanning for the vein. In other embodiments, the position detection system can be activated using a trigger. For example, the actuator assembly can be used to activate the position detection system

Adjusting the position of the handheld device 100 relative to the subject’s forearm 630 involves moving the device 100 over the forearm 630 until the position detection system indicates that the cartridge 102 is properly positioned over the vein 632. The position detection system can be associated with the handheld device and/or with the cartridge. For example, one or more optical sensors can be disposed on the handheld device and/or on the cartridge. Once it is determined that the cartridge 102 is properly positioned, the method further includes pressing the body-contacting interface 124 of the cartridge 102 onto the forearm to stabilize the vein. In some embodiments, as shown, e.g., in FIGS. 1, 2, 3A, and 3B, the body-contacting interface is a grooved channel formed of at least partially compressible material. As discussed above, the body-contacting interface 124 that is positioned over the patient’s skin is used to mechanically stabilize the vein in preparation for creating a puncture (e.g., lancing) the vein. This stabilization allows avoiding the possibility that the vein moves or slips out of the way of the puncture device (e.g., a needle or lancet), which would result in a miss and would require the process to be repeated.

Once the vein is “found” and stabilized, the method in accordance with embodiments of the present disclosure includes actuating the handheld device 100 thereby causing the cartridge 102 to deploy the puncture element 126 of the puncture assembly of the cartridge so that the puncture 126 element moves from an initial, retracted position to an extended, extended position to create a puncture in the vein. In some embodiments, the actuation is performed using an actuating assembly of the handheld device, by applying force to an actuation element disposed on a proximal handle of the handheld device. This can involve, for example, pressing the actuator element 109, or actuating another element, which can be different depending on the implementation of the device. The puncture is created to a certain depth within the vein, and, in some embodiments, the depth of the penetration of the puncture element 126 is controllable (e.g., using a depth control actuator or another suitable component). For example, in some embodiments, the puncture in the vein has a depth of no greater than 10 mm (e.g., or about 2 mm, or about 3 mm, or about 4 mm, or about 5 mm, or about 6 mm, or about 7 mm, or about 8 mm, or about 9 mm, or about 10 mm). In some embodiments, the puncture in the vein has a depth of no greater than 7 mm. In some embodiments, the puncture in the vein has a depth of about 7 mm.

The actuation of the actuator assembly to cause the puncture element 126 to create a puncture also causes the cartridge to withdraw a blood sample through the puncture from the vein to at least one blood storage container included in the cartridge, such as the blood storage containers 122a, 122b. The blood sample of a suitable volume can be acquired. For example, in some embodiments, the blood sample has a volume of at least 1 mL, or at least 1.5 mL, or at least 2 mL. In some embodiments, the blood sample has a volume of about 1 mL, or about 1.5 mL, or about 2 mL. In some embodiments, the blood sample has a volume of less than 1 mL, or less than 1.5 mL, or less than 2 ml. In some embodiments, the blood sample has a volume of at least 1 mL and less than 2 mL. In some embodiments, the blood sample has a volume of at least 0.5 mL or about 500 uL. The blood sample can be acquired in a time period that is less than two minutes, less than one minute, or less than 45 seconds, or less than 30 seconds.

Thus, the present devices and methods allow collecting blood faster than existing approaches to collecting blood without the need for phlebotomy. Those existing approaches pertain to extracting capillary blood in larger volumes, which is accomplished by creating several puncture wounds, as opposed to one. Some of the existing blood drawing approaches are meant to be self-administered, much like the finger prick method, but with a decreased user intervention in the collection process. The devices in those approaches can adhere to the skin (usually below the shoulder), with a press of a button, create the puncture wounds and collect the blood into an internal blood tube. These methods were shown to be able to collect about 200 µL of blood after several minutes of use, and the quality of the blood is typically poor, which limits these methods’ clinical utility and value. The devices and methods in accordance with the present disclosure may allow collecting a blood sample in less than one minute.

As discussed above, in some embodiments, pressure is applied to the puncture, which has the isolation chamber 128 (see FIGS. 3A and 3B) disposed therearound such that, as the blood is sucked into the cartridge, the skin around the puncture adopts a dome-like shape with the puncture (which is stretched open) at the apex. In some embodiments, the pressure can be negative pressure, such as constant negative pressure. The application of the negative pressure to the puncture assists in maintaining the puncture open during the blood withdrawal process. This also affects positively the quality of the withdrawn blood. In particular, by causing the puncture wound to remain open, an unhindered conduit is created and maintained for the blood to exit the subject’s body. Such unrestricted pathway allows decreasing the hemolysis (bursting of RBCs), and decreases the amount of stress that the platelets experience, hence delaying the coagulation process and thereby improving the quality of the collected sample. Keeping the puncture open allows reducing the amount of shear stress on the cells to minimize cell lysis and platelet activation. A pressure applied to a puncture can have any suitable value. In some embodiments, for example, a pressure of -4 psi can be applied to a puncture, to enable collection of about 1 mL of blood in less than a minute. The pressure can have other values as well, and other volumes of blood can be collected (e.g., less than 2 mL).

In some embodiments, the described handheld device can be used for administering blood or another liquid (e.g., without limitation, a therapeutic) into the blood stream. In such embodiments, a cartridge can, for example, have a vaccine or another therapeutic stored therein that can be administered to a subject when the cartridge is inserted into the device. Embodiments of the present disclosure are not limited to any specific type of therapeutic or another composition that can be administered using a handheld device and a cartridge in accordance with embodiments of the present disclosure.

The method of collecting the blood sample from the subject further includes, when the blood sample is received in the at least one blood storage container, actuating the handheld device thereby causing the puncture element to move from the extended position to the initial, retracted position. For example, the actuator assembly can be activated. FIG. 3B illustrates the puncture element in the extended position, while FIG. 3A illustrates the cartridge 102 is the initial, retracted position. FIGS. 3C and 3D illustrate examples of puncture elements that can be used. As discussed above, in some embodiments, the actuator assembly remains activated while the blood withdrawal takes place such that the puncture element, in the extended position, remains in the puncture that it created. In other implementations, however, the puncture element is extended to create a puncture in the subject’s skin to access blood through the puncture, and retracted once the puncture is created, such that the blood withdrawal occurs while the puncture element is in the initial, retracted position.

The device 100 can be configured to generate an indication that the desired volume of the blood sample is received in the blood storage containers. The indication can be provided, e.g., via the graphical user interface 300, or via another component(s). Also, in some embodiments, a desired volume of blood can be acquired automatically, such that the blood drawing assembly automatically stops the blood withdrawal once the desired amount is acquired. The puncture element can also move from the second, extended position to the initial, retracted position automatically, or using a trigger. For example, the actuator assembly (e.g., actuator 109) can be manipulated to cause the puncture element to move to the initial, retracted position.

After the blood sample is received in the at least one blood storage container, the cartridge is separated from the handheld device. This can be performed using a suitable trigger. The handheld device can have a release component or another feature that can be manipulated by the user to remove the cartridge from the device. In some implementations, the handheld device and/or the cartridge can be configured such that the cartridge can be separated from the handheld device only into a receptacle of a sample analyzer. A suitable trigger and/or features can be used for this purpose. For example, the cartridge can have one or more magnets and a receptacle can have one or more magnets, such that the cartridge can be released from the handheld device upon the interaction between the magnet(s). Any other features can be used that allow releasing the cartridge specifically into a receptacle of a sample analyzer. For example, a Bluetooth® pairing between the cartridge (or the device) and the receptacle or another portion of the sample analyzer can be used to determine that the cartridge is in proximity to the receptacle.

In some embodiments, after the cartridge is separated from the handheld device, the cartridge can be coupled with a sample collection device, or the cartridge can be transferred to an analyzer. The sample collection device may comprise a plurality of compartments each configured to fit a cartridge. In some embodiments, as discussed above, the cartridge can be separated from the handheld device to be transferred directly to the analyzer. Also, in some implementations, the cartridge is released from the handheld device only once the cartridge is coupled to or otherwise associated with a suitable compartment or receptacle of a sample collection device or an analyzer. In some implementations, as also discussed above, the analyzer can have features that allow acquiring blood from blood storage containers of the cartridge.

In some embodiments, the blood sample is removed from the cartridge, e.g., by accessing ports of the at least one blood storage container. The “spent” cartridge, which can be disposable, can then be disposed.

The handheld device is reusable. Accordingly, in some embodiments, after the cartridge (with the blood sample stored therein) is separated from the handheld device, a second cartridge can be inserted into the handheld device. The handheld device with the second cartridge inserted herein can be used for collecting a blood sample from a patient. Multiple disposable cartridges can be used with the handheld device.

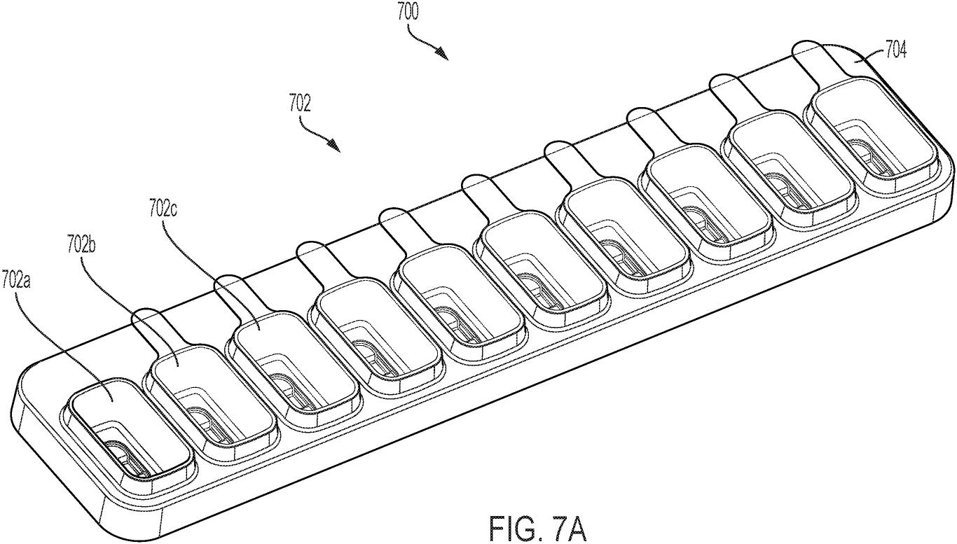

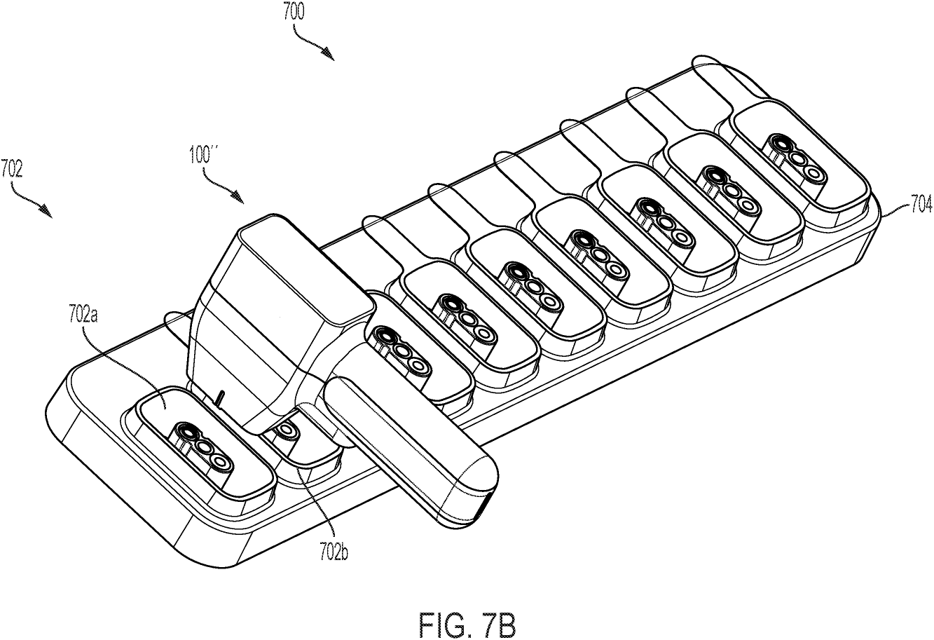

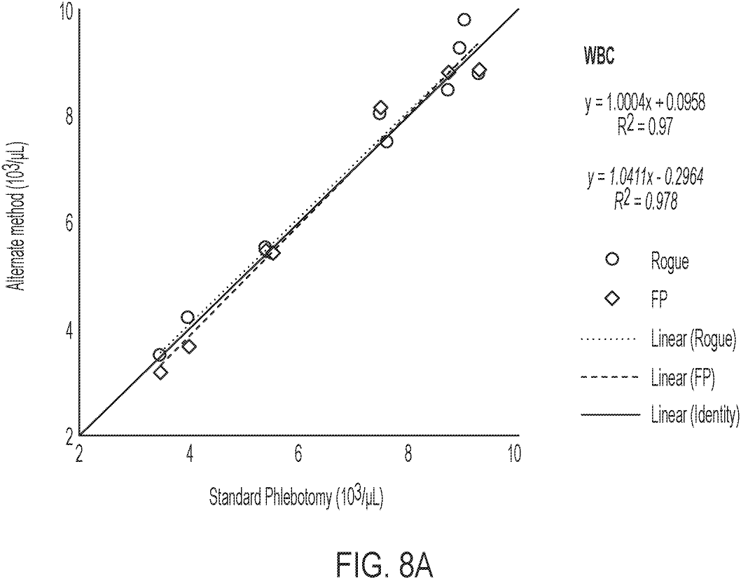

FIGS. 7A and 7B show a tray 700 having multiple compartments 702 (ten, in this example, though the tray can include any other number of compartments) formed in a body 704 of the tray 700, of which compartments 702a, 702b, and 702c are labeled. The compartments 702 of the tray 700 can each include a cartridge for use with a handheld device. The tray 700 can be manufactured in the form as shown in FIG. 7B, and a handheld device 100″ can be used to pick up a (new) cartridge from a respective compartment, as shown in FIG. 7B. FIG. 7B illustrates, by way of example, the device 100″ as it is picking up the cartridge from the compartment 702. After the cartridge is used to acquire a blood sample, the device 100″ can be used to pick up another cartridge from the tray’s compartment.

Additional information on embodiments of the present disclosure, as well as other relevant information, can be found in the Appendix submitted along with the present application.

### EXAMPLES

### Example 1

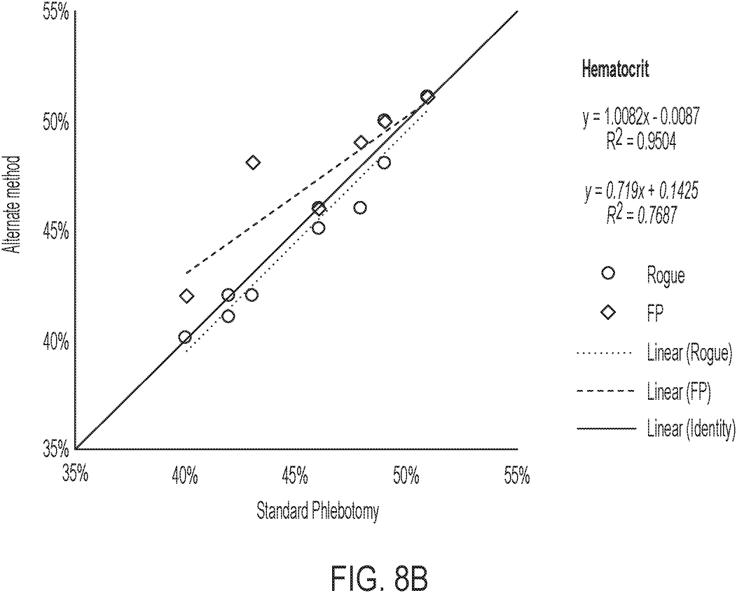

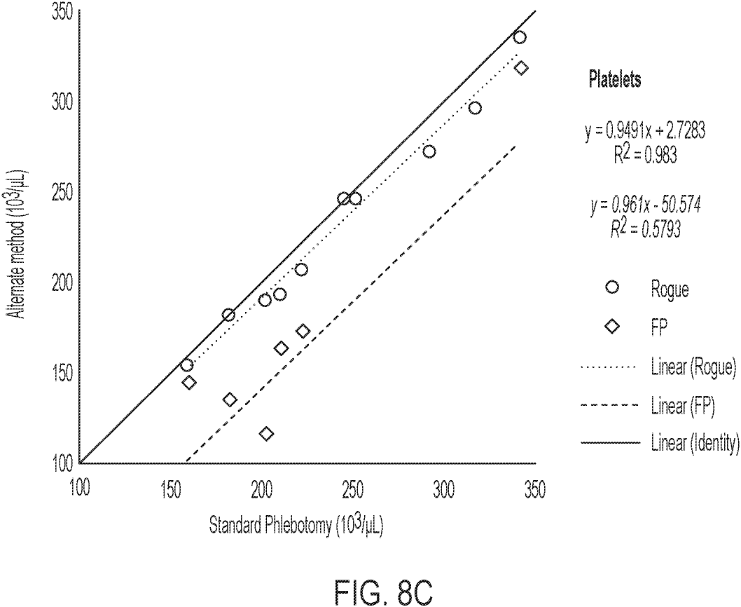

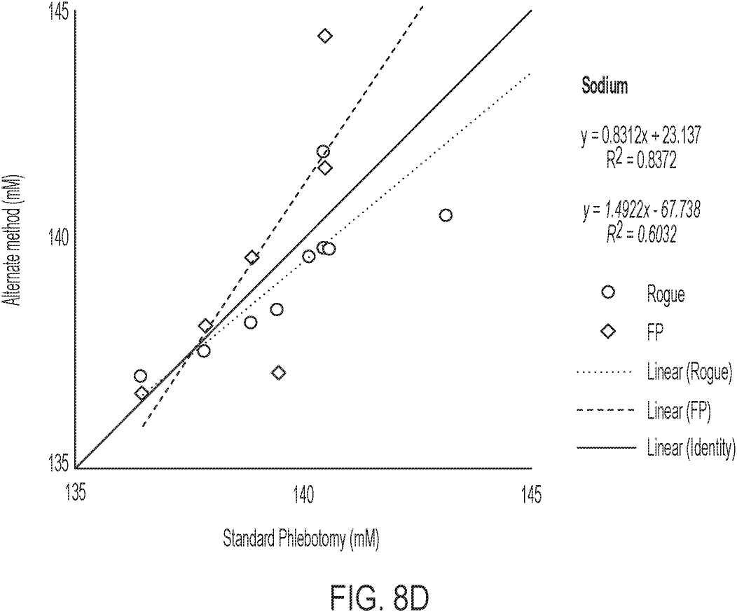

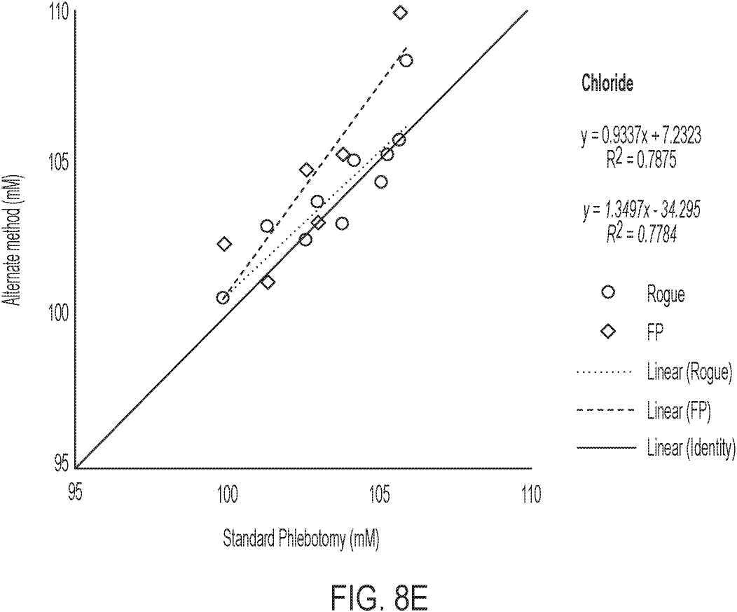

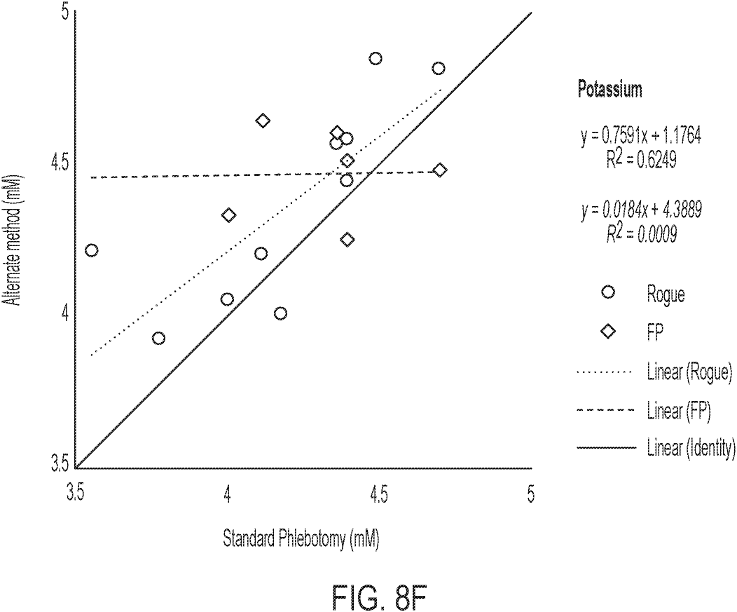

FIGS. 8A to 8F illustrate study results of measurements of several blood parameters, analyzed by FDA-approved clinical analyzers from Beckman Coulter (DxH 500 and AU 480). In the study, blood drawn from ten subjects was acquired using an approach of the present disclosure (“Rogue,” values and lines are shown as a dotted line) and the finger prick approach (“FP,” values and lines are shown as an evenly spaced dashed line), and concordance correlation coefficients were used to determine how well the measurements from these blood samples compare to measurements from blood samples acquired using the standard phlebotomy approach. FIGS. 8A to 8F illustrate results of measurements of various blood parameters such as WBC (while blood count) (FIG. 8A), Hematocrit (FIG. 8B), Platelets (FIG. 8C), Sodium (FIG. 8D), Chloride (FIG. 8E), and Potassium (FIG. 8F). FIGS. 8A-8F show measured values and linear regression (best-fit) line (for both Rogue and finger prick) for a respective blood parameter, as well as a linear (identity) line (shown as a solid line).

As shown in the table below, there is concordance between the present approach and phlebotomy. At the same time, concordance is not observed between data for the finger prick and phlebotomy approaches, for the most sensitive analytes (i.e., platelets and potassium).

Additional information on results of the conducted experiments is shown in the Appendix submitted along with the present application.

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties. The publications discussed herein are provided solely for their disclosure prior to the filing date of the present application. Nothing herein is to be construed as an admission that the present invention is not patentable in view of such publications.

As used herein, all headings are simply for organization and are not intended to limit the disclosure in any manner. The content of any individual section may be equally applicable to all sections.

What is claimed is:

1. A handheld device for collecting a blood sample from a subject, the handheld device comprising:
an actuator assembly;
a body housing the actuator assembly and having a proximal end and a cavity configured to releasably receive a cartridge to couple to the actuator assembly, the cartridge being configured to capture the blood sample from the subject;
a handle coupled to the proximal end of the body; and
a position detection system coupled to the body and having at least one sensor configured to determine a position of the cartridge relative to a target area of the subject when the cartridge is disposed within the cavity.

2. The handheld device of claim 1 further comprising:
the cartridge, the cartridge comprising:
a housing;
at least one blood storage container contained within the housing;
a distal body-contacting interface of the housing;
a puncture assembly contained within the housing and comprising a
puncture element moveable relative to the housing and contained within the
housing in an initial position and extending from the body-contacting interface in an extended position; and
a blood drawing assembly coupled configured to collect the blood sample from a puncture created in the target area by the puncture element in the extended position, wherein the blood drawing assembly delivers the blood sample to the at least one blood storage container.

3. The handheld device of claim 2, wherein the body-contacting interface is a grooved channel having a shape that corresponds to a shape of the target area of the subject.

4. The handheld device of claim 2, wherein the body-contacting interface comprises an opening that receives therethrough an isolation chamber coupled to the housing, the isolation chamber having a central opening that receives therethrough the puncture element in the extended position.

5. The handheld device of claim 2, wherein the blood drawing assembly is coupled to the puncture assembly.

6. The handheld device of claim 2, wherein the puncture element has a lumen extending therethrough and configured to deliver blood from the puncture to the at least one blood storage container.

7. The handheld device of claim 2, wherein the puncture element has at least one through opening.

8. The handheld device of claim 2, wherein the puncture element is a solid element.

9. The handheld device of claim 2, wherein the puncture element has or is associated with a sensor configured to detect when the puncture element encounters liquid.

10. The handheld device of claim 4, wherein the isolation chamber is retractable such that it is retracted proximally towards the housing when the puncture element moves from the initial position to the extended position.

11. The handheld device of claim 2, wherein an outer surface of the housing of the cartridge has at least one longitudinal groove extending from a proximal end of the housing towards a distal end of the housing and configured to mate with a corresponding protrusion formed on an inner surface of the cavity.

12. The handheld device of claim 2, wherein at least one blood storage container comprises first and second blood storage containers disposed on opposite sides of the puncture assembly.

13. The handheld device of claim 2, wherein the actuator assembly is configured to apply negative pressure to the target area while the blood drawing assembly draws the blood sample through the puncture in the target area.

14. The handheld device of claim 2, wherein the actuator assembly is configured to apply a vacuum pressure to the target area while the blood drawing assembly draws the blood sample through the puncture in the target area, and
wherein the actuator assembly is configured to increase and decrease the vacuum pressure one or more times.

15. The handheld device of claim 2, wherein at least one blood storage container comprises an access port for removing the blood sample from at least one blood storage container.

16. The handheld device of claim 2, wherein the blood drawing assembly comprises at least one fluid conduit configured to deliver the blood sample acquired through the puncture to the at least one blood storage container.

17. The handheld device of claim 2, wherein the actuator assembly is configured to be activated to cause the puncture element of the puncture assembly to puncture a skin surface of the subject at the target area and to cause a blood withdrawal assembly to collect the blood sample from the target area through a puncture in the target area into the cartridge.

18. The handheld device of claim 2, wherein the body-contacting interface is formed of at least partially compressible material.

19. The handheld device of claim 2 further comprising:
a depth control actuator coupled to the body and configured to be actuated to control a depth of the puncture to be created by the puncture element in the extended position.

20. The handheld device of claim 1, wherein the at least one sensor comprises first and second optical sensors positioned on a distal side of the body, proximate to opposite sides of the cavity.

21. The handheld device of claim 19, where the position detection system comprises a graphical user interface configured to present a graphical indication of the position of the cartridge relative to the target area.

22. The handheld device of claim 21, wherein the graphical user interface is disposed on a proximal side of the body.

23. The handheld device of claim 20, wherein the target area comprises a vein, and the graphical user interface displays an indictor indicating a position of the cartridge relative to the vein.

24. The handheld device of claim 1, wherein the actuator assembly comprises an actuator disposed on the handle and configured to be activated by a pressure applied thereto.

25. The handheld device of claim 1, wherein the position detection system is activated automatically when the handheld device detects that the cartridge is received in the cavity and coupled to the actuator assembly.

26. The handheld device of claim 1, wherein a portion of the body proximate to the cavity has curved skin contacting edges.

27-46. (canceled)
